(19) United States
(12) Patent Application Publication
(10) Pub. No.: US 2026/0034176 A1
(43) Pub. Date: Feb. 5, 2026

(54) T CELLS FOR USE IN TREATING RELAPSED OR REFRACTORY ACUTE MYELOID LEUKEMIA

(71) Applicant: TAKEDA PHARMACEUTICAL COMPANY LIMITED, Osaka (JP)

(72) Inventors: Cori ABIKOFF, Cambridge, MA (US); Kondala ATKURI, Cambridge, MA (US); Yue LIU, Cambridge, MA (US)

(21) Appl. No.: 19/351,125

(22) Filed: Oct. 6, 2025

Related U.S. Application Data
(63) Continuation of application No. PCT/IB2024/053367, filed on Apr. 5, 2024.
(60) Provisional application No. 63/457,937, filed on Apr. 7, 2023.

(51) Int. Cl. A61K 35/17

(52) U.S. Cl. CPC A61K 35/17

(57) ABSTRACT

The present disclosure provides, among other things, methods for treating relapsed or refractory acute myeloid leukemia by administering to a subject in need thereof a therapeutically effective amount of an allogeneic composition comprising VDelta1+ (Vδ1+) gamma delta (γδ) T cells such that one or more symptoms or biomarkers is improved after treatment. The present disclosure also provides suitable doses of compositions comprising allogeneic Vδ1+gamma delta (γδ) T cells for administration to a subject suffering from relapsed or refractory AML. In some embodiments, the Vδ1+gamma delta (γδ) T cells are untransduced.

### CROSS-REFERENCE TO RELATED APPLICATIONS

This application is a Continuation of International Application No. PCT/IB2024/053367 filed on Apr. 5, 2024, which claims priority to, and the benefit of, U.S. Provisional Patent Application No. 63/457,937 filed on Apr. 7, 2023, the contents of each of which are herein incorporated by reference in their entireties.

### BACKGROUND

Acute myeloid leukemia (AML) is a type of blood cancer that affects adults and children, and is characterized by infiltration of bone marrow and other tissues by clonally proliferative immature myeloid cells. About 20,050 cases of acute myeloid leukemia are diagnosed annually (NCI Surveillance Epidemiology and End Results Program, SEER). Current therapeutic strategies include chemotherapy, allogeneic stem cell transplantation and select targeted therapies, however, treating AML remains a clinical challenge due to resistance to chemotherapy and a significant proportion of treated patients developing relapsed or refractory disease.

AML has a poor survival rate among the elderly (age 65 or older), and an average overall 5-year survival rate of approximately 30%, mostly due to resistance to standard treatment. For example, chemotherapy with a combination of cytarabine with an anthracyclin drug, although effective at inducing complete remissions, ultimately selects for chemoresistant clones that drive refractory relapses. The median cumulative incidence of relapse is 29.4% after stem cell transplant and 46.8% after induction chemotherapy and a substantial portion of patients develop refractory or relapsed AML with poor prognosis. (Esther N. O. et al. (2021), Am J Blood Res, 11(4): 325-360).

The presence of gamma delta T cells have been shown to have a positive correlation with prognosis in a number of solid and haematological cancers (Deniger, D. C. et al., Clin. Cancer Res. (2014), 20(22): 5708-19; Gentles A. J. et al., Nat. Med. (2015), 21(8): 938-945). There is a need in the field for a treatment option for AML, in particular relapsed or refractory AML, that is both safe and effective.

### SUMMARY OF THE INVENTION

The present disclosure provides, among other things, methods of treating relapsed or refractory acute myeloid leukemia by administering to a subject in need thereof a therapeutically effective amount of a composition (e.g., an allogeneic composition) comprising Vδ1+ gamma delta (γδ) T cells such that one or more symptoms or biomarkers is improved after treatment. The present disclosure also provides suitable doses of compositions comprising allogeneic Vδ1+ gamma delta (γδ) T cells for administration to a subject suffering from relapsed or refractory AML.

Vδ1+ γδ T cells are an enriched subset of γδ T cells, that predominantly comprise Vδ1+ γδ T cells that recognize malignant cells through expression of a diverse repertoire of natural cytotoxicity receptors (NCRs) that interact with stress ligands, pAgs, lipid Ags and many other non-peptide molecules specifically upregulated on diseased cells. While αβ T cells require the MHC-antigen axis for activation to occur, gamma delta T lymphocytes do not require major histocompatibility complex (MHC)-mediated antigen presentation to exert their cytotoxic effect, thus the initial responsiveness of γδ T cells precede that of αβ T cells as they can directly recognize and are activated by molecular patterns of dysregulation on cancer cells. Without wishing to be bound by any particular theory, it is contemplated that non-MHC-restricted immunomodulating and antineoplastic activity of Vδ1+ γδ T cells (GDX012) of the present disclosure provides a novel allogeneic cell therapy for treatment of relapsed or refractory AML, which are particularly challenging to treat. The inventors of the present application have developed an ‘off-the-shelf’ immunotherapy for relapsed or refractory AML which is not resolved by other modes of treatment. GDX012 therapy has also been described in WO2021/186137, the contents of which are incorporated by reference herein in their entirety.

When administered systemically, for example, by intravenous infusion, GDX012 shows homing to the bone marrow and is detected for at least 28 days. Without wishing to be bound by any particular theory, it is contemplated that in addition to high cytotoxic activity against AML blasts, GDX012 also has the capacity to home to and persist in peripheral blood and the bone marrow, exhibiting prolonged cytotoxic effect within target tissue with a single dose in treating relapsed or refractory AML. The Vδ1+ γδ T cells (GDX012) of the present disclosure thus provide safe and efficacious therapy for relapsed or refractory AML. In some embodiments, GDX012 is administered in multiple doses and/or used in combination with other treatments.

In some aspects, the administration of GDX012 is preceded by lymphodepletion therapy. In some embodiments, cytarabine and fludarabine are administered as lymphodepletion therapy. Cytarabine and fludarabine are efficacious in combination as lymphodepletion agents. Without wishing to be bound by any particular theory, cytarabine is intracellularly converted to an active metabolite, ara-C 5′-triphosphate (ara-CTP). Fludarabine potentiates the intracellular accumulation of ara-CTP, thereby increasing the cytotoxic effect of cytarabine.

While the use of Vδ2+ T cells in cancer treatments have been explored, including, for example, in relapsed or refractory AML (Vydra et al., Clinical Lymphoma (2023), Myeloma and Leukemia), the clinical use of Vδ1+ T cells has been challenging due to their relatively low abundance of less than about 0.5% among peripheral blood lymphocytes. The present disclosure provides a method of treating relapsed or refractory AML by administering an allogeneic composition of Vδ1+ T cells (GDX012) obtained to a high yield for therapeutic use, in doses safe and suitable for administration to a subject suffering from relapsed or refractory AML. Relapsed or refractory AML is typically associated with a higher disease load and larger numbers of leukemic blasts in bone marrow and peripheral blood, as compared to, for example, a Measurable Residual Disease (MRD) state. The present disclosure provides efficacious doses of GDX012 for resolving or minimizing one or more symptoms or biomarkers of relapsed or refractory acute myeloid leukemia.

In some aspects, provided herein is a method of treating acute myeloid leukemia by administering to a subject in need thereof a therapeutically effective amount of a composition comprising Vδ1+ gamma delta (γδ) T cells, wherein the acute myeloid leukemia is relapsed or refractory.

In some embodiments, wherein one or more symptoms or biomarkers is improved after the administration.

In some embodiments, the gamma delta T cells are untransduced.

In some embodiments, the composition comprises at least about 60%, 65%, 70%, 75%, 80%, 85%, 90%, 95%, or about 99% gamma delta T cells relative to total live cells.

In some embodiments, the composition comprises at least 50% Vδ1+ γδ T cells relative to total live cells.

In some embodiments, the composition comprises at least 70% Vδ1+ γδ T cells relative to total live cells.

In some embodiments, the composition comprises at least 90% Vδ1+ γδ T cells relative to total live cells.

In some embodiments, the composition comprises at least 99% Vδ1+ γδ T cells relative to total live cells.

In some embodiments, the composition comprises less than 5% residual Vδ2+ cells.

In some embodiments, the composition comprises less than 0.5% residual Vδ2+ cells.

In some embodiments, the composition comprises less than 0.1% residual Vδ2+ cells.

In some embodiments, the composition comprises no residual Vδ2+ cells.

In some embodiments, the subject has previously been treated with chemotherapy.

In some embodiments, the subject has received at least two courses of intensive induction chemotherapy. In some embodiments, the subject has received two courses of intensive induction chemotherapy. In some embodiments, the subject has received three courses of intensive induction chemotherapy. In some embodiments, the subject has received four courses of intensive induction chemotherapy. In some embodiments, the subject has received five courses of intensive induction chemotherapy.

In some embodiments, the subject has about 5% leukemic blasts in bone marrow after (CR), Complete Remission with Partial Hematologic Recovery (CRh), Complete Remission with Incomplete Hematologic Recovery (CRi) or Morphologic Leukemia-free State (MLFS). In some embodiments, the subject has greater than about 5%, greater than about 10%, greater than about 20%, greater than about 25%, greater than about 30%, greater than about 35%, greater than about 40%, greater than about 45%, greater than about 50% leukemic blasts in bone marrow after Complete Remission (CR), Complete Remission with Partial Hematologic Recovery (CRh), Complete Remission with Incomplete Hematologic Recovery (CRi) or Morphologic Leukemia-free State (MLFS). In some embodiments, the subject has between 5% and 50% leukemic blasts in bone marrow. In some embodiments, the subject has greater than about 10% leukemic blasts in bone marrow. In some embodiments, the subject has greater than about 15% leukemic blasts in bone marrow. In some embodiments, the subject has greater than about 20% leukemic blasts in bone marrow. In some embodiments, the subject has greater than about 25% leukemic blasts in bone marrow. In some embodiments, the subject has greater than about 30% leukemic blasts in bone marrow. In some embodiments, the subject has greater than about 35% leukemic blasts in bone marrow. In some embodiments, the subject has greater than about 40% leukemic blasts in bone marrow. In some embodiments, the subject has greater than about 45% leukemic blasts in bone marrow. In some embodiments, the subject has greater than about 50% leukemic blasts in bone marrow.

In some embodiments, the subject has about 5% leukemic blasts in peripheral blood after (CR), Complete Remission with Partial Hematologic Recovery (CRh), Complete Remission with Incomplete Hematologic Recovery (CRi) or Morphologic Leukemia-free State (MLFS). In some embodiments, the subject has greater than about 5%, greater than about 10%, greater than about 20%, greater than about 25%, greater than about 30%, greater than about 35%, greater than about 40%, greater than about 45%, greater than about 50% leukemic blasts in peripheral blood after Complete Remission (CR), Complete Remission with Partial Hematologic Recovery (CRh), Complete Remission with Incomplete Hematologic Recovery (CRi) or Morphologic Leukemia-free State (MLFS). In some embodiments, the subject has between 5% and 50% leukemic blasts in peripheral blood. In some embodiments, the subject has greater than about 5% leukemic blasts in peripheral blood. In some embodiments, the subject has greater than about 10% leukemic blasts in peripheral blood. In some embodiments, the subject has greater than about 15% leukemic blasts in peripheral blood. In some embodiments, the subject has greater than about 20% leukemic blasts in peripheral blood. In some embodiments, the subject has greater than about 25% leukemic blasts in peripheral blood. In some embodiments, the subject has greater than about 30% leukemic blasts in peripheral blood. In some embodiments, the subject has greater than about 35% leukemic blasts in peripheral blood. In some embodiments, the subject has greater than about 40% leukemic blasts in peripheral blood. In some embodiments, the subject has greater than about 45% leukemic blasts in peripheral blood. In some embodiments, the subject has greater than about 50% leukemic blasts in peripheral blood.

In some embodiments, the therapeutically effective amount comprises about 8×10¹⁰, 4×10¹⁰, 8×10⁹, 4×10⁹, 2.4×10⁹, 1.2×10⁹, 8×10⁸, 4×10⁸, 8×10⁷ or 4×10⁷ live T cells.

In some embodiments, the therapeutically effective amount comprises about 8×10⁹ live T cells.

In some embodiments, the therapeutically effective amount comprises about 4×10⁹ live T cells.

In some embodiments, the therapeutically effective amount comprises about 2.4×10⁹ live T cells.

In some embodiments, the therapeutically effective amount comprises about 1.2×10⁹ live T cells.

In some embodiments, the therapeutically effective amount comprises about 8×10⁸ live T cells.

In some embodiments, the therapeutically effective amount comprises about 4×10⁸ T cells.

In some embodiments, the therapeutically effective amount comprises less than about 4×10⁸ T cells.

In some embodiments, the therapeutically effective amount comprises less than about 5×10⁴ alpha beta T cells.

In some embodiments, the therapeutically effective amount comprises less than about 1×10⁴ alpha beta T cells.

In some embodiments, the gamma delta T cells do not express a chimeric antigen receptor (CAR).

In some embodiments, the gamma delta T cells express a chimeric antigen receptor (CAR).

In some embodiments, the composition is administered intravenously, transarterially, subcutaneously, intradermally, intratumorally, intranodally, intramedullary, intramuscularly, or intraperitoneally.

In some embodiments, the composition is administered intravenously.

In some embodiments, the composition is administered transarterially. In some embodiments, the composition is administered subcutaneously. In some embodiments, the composition is administered intradermally. In some embodiments, the composition is administered intratumorally. In some embodiments, the composition is administered intranodally. In some embodiments, the composition is administered intramedullary. In some embodiments, the composition is administered intramuscularly. In some embodiments, the composition is administered intraperitoneally.

In some embodiments, the composition is administered in one or more doses.

In some embodiments, the composition is administered in one dose.

In some embodiments, the composition is administered once a week, once a month or once in two months. In some embodiments, the composition is administered once a week. In some embodiments, the composition is administered once in two months. In some embodiments the composition is administered once every two weeks.

In some embodiments, the composition is administered once a month.

In some embodiments, the composition is administered for a period of 1 month, 2 months, 4 months, 6 months, 12 months, 14 months, 18 months or 24 months. In some embodiments, the composition is administered for a period of 1 month. In some embodiments, the composition is administered for a period of 2 months. In some embodiments, the composition is administered for a period of 4 months. In some embodiments, the composition is administered for a period of 6 months. In some embodiments, the composition is administered for a period of 12 months. In some embodiments, the composition is administered for a period of 14 months. In some embodiments, the composition is administered for a period of 18 months. In some embodiments, the composition is administered for a period of 24 months.

In some embodiments, the composition is administered for a period of greater than 24 months.

In some embodiments, the chemotherapy comprises administration of mitoxantrone, etoposide, cytarabine or anthracycline. In some embodiments, the chemotherapy comprises administration of mitoxantrone. In some embodiments, the chemotherapy comprises administration of etoposide. In some embodiments, the chemotherapy comprises administration of cytarabine. In some embodiments, the chemotherapy comprises administration of anthracycline.

In some embodiments, the chemotherapy comprises administration of fludarabine and cyclophosphamide.

In some embodiments, the chemotherapy comprises administration of fludarabine and cytarabine.

In some embodiments, the chemotherapy comprises administration of 7 days of cytarabine and 3 days of anthracycline.

In some embodiments, the chemotherapy comprises administration of at least two cycles of hypomethylating agent. In some embodiments, the chemotherapy comprises administration of two cycles of hypomethylating agent. In some embodiments, the chemotherapy comprises administration of three cycles of hypomethylating agent. In some embodiments, the chemotherapy comprises administration of four cycles of hypomethylating agent.

In some embodiments, the chemotherapy comprises multiple cycles of monotherapy with a hypomethylating agent. In some embodiments, the chemotherapy comprises at least four cycles of monotherapy with a hypomethylating agent. In some embodiments, the chemotherapy comprises five cycles of monotherapy with a hypomethylating agent. In some embodiments, the chemotherapy comprises administration of greater than five cycles of monotherapy with a hypomethylating agent.

In some embodiments, cytarabine is administered with venetoclax, fludarabine or other B-cell lymphoma 2 (BCL2) inhibitors. In some embodiments, cytarabine is administered with venetoclax. In some embodiments, cytarabine is administered with fludarabine. In some embodiments, cytarabine is administered with one or more B-cell lymphoma 2 inhibitors.

In some embodiments, the subject is at least 2 years old.

In some embodiments, the subject is at least 12 years old.

In some embodiments, the subject is at least 18 years old.

In some embodiments, the one or more symptoms or biomarkers that is improved after treatment is that bone marrow blasts are substantially absent.

In some embodiments, the one or more symptoms or biomarkers that is improved after treatment is that bone marrow blasts are less than about 5%.

In some embodiments, the one or more symptoms or biomarkers that is improved after treatment is that bone marrow blasts and/or peripheral blood blasts are from between 5% to 25%.

In some embodiments, the one or more symptoms or biomarkers that is improved after treatment is that bone marrow blasts are less than about 10%.

In some embodiments, the one or more symptoms or biomarkers after treatment that is improved is that bone marrow blasts are less than about 15%.

In some embodiments, the one or more symptoms or biomarkers after treatment that is improved is that bone marrow blasts are less than about 20%.

In some embodiments, the one or more symptoms or biomarkers after treatment that is improved is that bone marrow blasts are less than about 25%.

In some embodiments, the one or more symptoms or biomarkers that is improved after treatment is that peripheral blood blasts are substantially absent.

In some embodiments, the one or more symptoms or biomarkers that is improved after treatment is that peripheral blood blasts are less than about 5%.

In some embodiments, the one or more symptoms or biomarkers that is improved after treatment is that peripheral blood blasts are less than about 10%.

In some embodiments, the one or more symptoms or biomarkers that is improved after treatment is that peripheral blood blasts are less than about 15%.

In some embodiments, the one or more symptoms or biomarkers that is improved after treatment is that peripheral blood blasts are less than about 20%.

In some embodiments, the one or more symptoms or biomarkers that is improved after treatment is that peripheral blood blasts are less than about 25%.

In some embodiments, the one or more symptoms or biomarkers that is improved after treatment is decrease of pretreatment bone marrow blast percentage by at least 50% based on European Leukemia Net (ELN) 2022 response criteria for AML.

In some embodiments, the one or more symptoms or biomarkers that is improved after treatment is the absence of extramedullary disease.

In some embodiments, the one or more symptoms or biomarkers that is improved after treatment is absolute neutrophil count (ANC) of 0.5×10⁹/Liters or greater.

In some embodiments, the one or more symptoms or biomarkers that is improved after treatment is absolute neutrophil count (ANC) of 1.0×10⁹/Liters or greater.

In some embodiments, the one or more symptoms or biomarkers that is improved after treatment is platelet count of 50×10⁹/Liters or greater.

In some embodiments, the one or more symptoms or biomarkers that is improved after treatment is platelet count of 100×10⁹/Liters or greater.

In some embodiments, after treatment Measurable Residual Disease (MRD) is less than about 0.1%.

In some embodiments, MRD is measured by flow cytometry of bone marrow cells and/or blood. In some embodiments, MRD is measured by flow cytometry of bone marrow cells. In some embodiments, MRD is measured by flow cytometry of blood.

Any numerals used in this application with or without the terms “about” or “approximately” are meant to cover any normal fluctuations appreciated by one of ordinary skill in the relevant art.

Other features, objects, and advantages of the present disclosure are apparent in the detailed description that follows. It should be understood, however, that the detailed description, while indicating embodiments of the present disclosure, is given by way of illustration only, not limitation. Various changes and modifications within the scope of the disclosure will become apparent to those skilled in the art from the detailed description.

### Definitions

In order for the present disclosure to be more readily understood, certain terms are first defined below. Additional definitions for the following terms and other terms are set forth throughout the specification.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, the term refers to a range of values that fall within 10% of the stated reference value. In some embodiments, the term refers to a range of values that fall within 5% of the stated reference value. The term “between” includes the values of the specified boundaries and all intervening values and fractions.

Expanded population of Vδ1+ T cells: As used herein, “expanded” or “expanded population of Vδ1+ T cells” includes populations of cells which are larger or contain a larger number of cells than a non-expanded population. Such populations may be large in number, small in number or a mixed population with the expansion of a proportion or particular cell type within the population. It will be appreciated that the term “expansion step” refers to processes which result in expansion or an expanded population. Thus, expansion or an expanded population may be larger in number or contain a larger number of cells compared to a population which has not had an expansion step performed or prior to any expansion step. It will be further appreciated that any numbers indicated herein to indicate expansion (e.g. fold-increase or fold-expansion) are illustrative of an increase in the number or size of a population of cells or the number of cells and are indicative of the amount of expansion.

Enriched population of Vδ1+ T cells: As used herein, enriched population refers to an allogeneic composition of Vδ1+ cells depleted of αβ cells and comprising less than 5%, less than 0.5% or less than 0.1% residual Vδ2+ cells. In some embodiments, at least 90%, at least 95% or at least 99% of cells are Vδ1+ γδ T cells. In some embodiments, between 99.1% to 99.9% of cells are Vδ1+ γδ T cells. In some other embodiments, the Vδ1+ T cells comprises at least at least 60%, at least 70%, at least 80%, at least 90% of Vδ1+ γδ T cells relative to total live cells.

Interleukin-4-like activity: As used herein, a growth factor having “interleukin-4-like activity” means any compound that has the same activity as IL-4 with respect to its ability to promote similar physiological effects on γδ T cells in culture and includes, but is not limited to, IL-4 and IL-4 mimetics, or any functional equivalent of IL-4. The physiological effects promoted by IL-4 on γδ T cells have been shown to include the decrease of NKG2D and NCR expression levels, the inhibition of cytotoxic function and improved selective survival. IL-4 has also been shown to significantly inhibit the secretion of pro-inflammatory cytokines, including IFN-γ, TNF-α, from activated TCRγδ+ T cells.

Interleukin-15-like activity: As used herein, a growth factor having “interleukin-15-like activity” means any compound that has the same activity as IL-15 with respect to its ability to promote similar physiological effects on γδ T cells in culture and includes, but is not limited to, IL-15 and IL-15 mimetics, or any functional equivalent of IL-15, including IL-2 and IL-7. The physiological effects promoted by IL-15, IL-2 and IL-7 on cultured γδ T cells include the induction of cell differentiation towards a more cytotoxic phenotype, such as the upregulation of NKG2D and natural cytotoxicity receptors (NCR) (NKp30 and NKp44) expression levels, increased anti-tumour cytotoxic function and increased production of pro-inflammatory cytokines, such as IFN-γ.

Measurable physiological effect: As used herein, “measurable physiological effect” refers to any measurable change in a target cells' physiological state according to standard definitions. For example, changes in a cell's physiological state can be detected by changes in their activation state (recognized by the upregulation or downregulation of the expression levels of the early-activation cell marker CD69); or detected by changes in their differentiation state (recognized by the upregulation or downregulation of NKG2D or natural cytotoxicity receptors NCRs that mediate NK cell activation), a few hours or a few days after contact with such cytokines. A measurable physiological effect may also be a change in the cell's proliferation rate, as measured by amine-reactive dye CFSE (carboxyfluorescein diacetate, succinimidyl ester) staining or by other techniques known in the art.

Leukemic blast: Blast cells are abnormal immature white blood cells that multiply uncontrollably in a subject with acute myeloid leukemia, filling up the bone marrow, and preventing production of other cells important for survival, namely red blood cells and platelets. Blast cells found in bone marrow or peripheral blood in acute myeloid leukemia are termed ‘leukemic blast’ and is a measure of disease.

Measurable (or Minimal) Residual Disease (MRD): As used herein, minimal or, more appropriately, measurable residual disease (MRD) refers to an objective measure of AML remission by sensitive methods that permit determination of very low numbers of cancerous blasts, as well as distinguish between cancerous blasts and healthy blasts. For example, patients who achieve complete remission according to morphologic assessment alone may harbor some residual cancer cells in the bone marrow, with 30%-40% testing positive for MRD. MRD denotes the presence of leukemia cells down to levels of 1:10⁴ to 1:10⁶ white blood cells (WBCs), compared with 1:20 in morphology-based assessments. MRD detection in AML is used to establish a deeper remission status, to refine outcome prediction and inform post-remission treatment, to identify impending relapse and enable early intervention, to allow more robust posttransplant surveillance, and to use as a surrogate end point for drug testing.

Morphologic Leukemia-free State (MLFS): As used herein, “morphologic leukemia-free state (MLFS)” is characterized by bone marrow blasts of less than 5%, absence of circulating blasts, absence of extramedullary disease, no hematologic recovery is required. Marrow is not merely aplastic, bone marrow spicules are present, at least 200 cells are enumerated in aspirate and cellularity is at least 10% in a biopsy.

Refractory Acute Myeloid Leukemia (AML): “Refractory AML” refers to leukemia that did not respond to treatment. Complete remission (CR) is not reached because the chemotherapy drugs do not kill enough leukemia cells, neither is CRh or CRi reached at the response landmark, i.e, after 2 courses of intensive induction treatment or a defined landmark, eg, 180 d after commencing less-intensive therapy

Relapsed Acute Myeloid Leukemia (AML): “Relapsed AML”, or recurrent acute myeloid leukemia (AML) refers to leukemia that has come back after treatment and remission. Relapsed AML is associated with one or more symptoms and biomarkers such as bone marrow blasts of 5% or greater than 5%; or reappearance of blasts in the blood in at least two peripheral blood samples at least one week apart; or development of extramedullary disease.

Remission: As used herein, the term “remission” means that the signs and symptoms of cancer are reduced. Remission can be partial or complete. Measurable (or minimal) residual disease (MRD); cancer cells not destroyed by treatment) is a more sensitive measure of remission.

Complete Remission (CR): In a complete remission (CR), all signs and symptoms of acute myeloid leukemia have disappeared, for example, the bone marrow contains fewer than 5% blast cells and circulating blasts are absent. Extramedullary disease is absent. Blood cell counts return to within normal limits, for example, neutrophil count of 1.0×10⁹/L (1,000/μL) or greater and platelet count of at least 100×10⁹/L [100,000/μL]). If a person remains in complete remission for 5 years or more, the cancer is sometimes considered cured.

Partial Remission (PR): As used herein, in a partial remission (PR), all hematologic criteria of CR are met, but there is a decrease of bone marrow blast percentage to 5% to 25%, and decrease of pre-treatment bone marrow blast percentage by at least 50%.

Complete Remission with Partial Hematologic Recovery (CRh): The term “CR with partial hematologic recovery (CRh)” has been introduced for patients with morphologic bone marrow blast clearance and partial recovery of both neutrophils (at least 0.5×10⁹/L [500/μL]) and platelets (at least 50×10⁹/L [50,000/μL]) because those represent clinical benefit to the patient; other CR criteria need to be met.

Complete Remission with Incomplete Hematologic Recovery (CRi): The term “CR with incomplete hematologic recovery (CRi)” has been introduced because CR criteria are met except for residual neutropenia of less than 1.0×10⁹/L (1,000/μL) or thrombocytopenia of less than 100×10⁹/L (100,000/μL).

Duration of Response: As used herein, “Duration of Response (DOR)” is defined as the time from the date of first documented CR, CRh, or CRi to the date of relapse or death.

Event-free Survival (EFS): As used herein, “Event-free Survival” is defined as the time from the date of first dose administration to the date of treatment failure, relapse or death, whichever comes first.

Overall Survival (OS): As used herein, “Overall Survival” is defined as the time from the date of the first dose administration to the date of death.

Subject or Patient: As used herein, “subject” or “patient” refers to an individual suffering from acute myeloid leukemia, wherein the acute myeloid leukemia is relapsed or refractory.

Symptom: As used herein, “symptom” includes, but is not limited to, the following: weight loss, fatigue, fever, night sweats, loss of appetite, weakness, feeling cold, feeling dizzy or lightheaded, headaches, pale skin, shortness of breath, bruising, excess bleeding, frequent or severe nosebleeds, bleeding or clotting problems, headache, slurred speech, equilibrium, blurred vision, facial numbness, enlarged lymph nodes, bone or joint pain and any combinations thereof.

Therapeutically effective amount: As used herein, the term “therapeutically effective amount” means an amount effective, at dosages, frequency of administration and for duration of time necessary to achieve the desired results such that one or more symptoms or biomarkers is improved after treatment.

### DETAILED DESCRIPTION

The present disclosure provides, among other things, methods of treating relapsed or refractory acute myeloid leukemia by administering to a subject in need thereof a therapeutically effective amount of a composition (e.g., an allogeneic composition) comprising Vδ1+ gamma delta (γδ) T cells such that one or more symptoms or biomarkers is improved after treatment. The present disclosure also provides suitable doses of compositions comprising allogeneic Vδ1+ gamma delta (γδ) T cells for administration to a subject with a high disease load (e.g., greater than or equal to 5%, 10%, or 20% leukemic blasts, or resistant leukemic blasts) as in patients suffering from relapsed or refractory acute myeloid leukemia.

### Acute Myeloid Leukemia

Acute myeloid leukemia (AML) is a cancer affecting adults and children, characterized by clonal expansion of proliferative immature myeloid cells and infiltration of peripheral blood, bone marrow and other tissues. It is caused when either the myeloid stem cells produce abnormal myeloblasts which do not become healthy white blood cells or too many myeloid stem cells become abnormal red blood cells or platelets. As a result, leukemic blasts, or immature cell forms, accumulate in the bone marrow, peripheral blood, and occasionally in other tissues, and the production of normal red blood cells, platelets, and mature granulocytes is reduced.

Current therapeutic strategies include chemotherapy, allogeneic stem cell transplantation and novel targeted therapies in select cases. However, a significant proportion of patients exhibit relapsed or refractory disease.

Relapsed, or recurrent, acute myeloid leukemia (AML) refers to leukemia that has come back after treatment and remission. Relapse could occur due to, for example, a chemo-sensitive disease that was partially treated and recurs with additional mutations; a subclone, derived from the same founder clone as the predominant clone, initially present at low frequency but that undergoes clonal proliferation during treatment due to decreased chemotherapy sensitivity; or de novo generation of AML due to toxicity from treatment, for example, in late relapses that occur three or more years after achieving initial complete remission.

Refractory AML refers to leukemia that did not respond to treatment. Complete remission is not reached because the chemotherapy drugs do not kill enough leukemia cells.

Patients with measurable residual disease (MRD) comprise low disease loads of leukemic blasts, typically in the range of 10,000 to 100,00,00 bone marrow or peripheral blood (PB) leukemic cells. Both relapsed and refractory AML are typically associated with higher disease loads of leukemic cells and are particularly challenging to treat.

Chemotherapy is often used as a treatment modality for relapsed or refractory disease. Chemotherapy uses anticancer drugs to destroy cancer cells. Chemotherapy for AML can be divided into 3 phases: induction, post-remission consolidation, and maintenance. During induction therapy, which is the first period of treatment after a person's diagnosis, the goal is complete remission (CR). A person has a CR when blood counts have returned to normal, leukemic cells are not found in a bone marrow sample when examined under the microscope and there are no longer any signs and symptoms of AML. A second stage of therapy is post-remission consolidation, when a variety of different drugs are used to destroy AML cells that remain in small amounts that cannot be detected by medical tests.

Treatment includes repeating cycles of the same or similar drugs that were used in induction treatment if the complete remission was longer than one year. Similar or higher doses of the drugs may be used. Repeated courses of cytarabine for 7 days and anthracycline for 3 days, for example, daunorubicin, doxorubicin, idarubicin, mitoxantrone are administered for 7 days. Other types of chemotherapy combinations for relapsed or refractory AML include fludarabine (Fludara), cytarabine and filgrastim (Neupogen) (FLAG); mitoxantrone (Novantrone), etoposide (Vepesid) and cytarabine (MEC); high-dose cytarabine and mitoxantrone; high-dose etoposide and cyclophosphamide; cytarabine, daunorubicin and etoposide, clofarabine and cytarabine with or without filgrastim. However, many patients with relapsed or refractory AML cannot tolerate intensive chemotherapy. Less intensive chemotherapeutic regimens include, for example, azacitidine with or without venetoclax, decitabine with or without venetoclax and clofarabine with or without cytarabine. In some embodiments, fludarabine and cyclophosphamide are administered. AML will almost certainly recur if no further treatment is given after a complete remission. If acute myeloid leukemia spreads to the CNS, chemotherapy has to be provided intrathecally. Sometimes radiation therapy is also provided, for example, along with intrathecal chemotherapy. Radiation therapy uses high-energy rays or particles to destroy cancer cells.

Targeted therapy uses drugs to target specific molecules, for example, proteins on cancer cells to stop the growth and spread of cancer. For example, gilteritinib is used to treat AML with the FLT3 mutation, gemtuzumab ozogamicin is used to treat AML with a CD33 marker, enasidenib is used to treat AML due to a mutated IDH2 gene. However, targeted therapy relies on specific targets, and cancer cells can also become resistant to targeted therapy, for example, when the target molecule itself changes, and the targeted therapeutic agent is not able to interact with it, or when cancer cells are able to grow in new ways that do not depend on the target.

For some patients, bone marrow/stem cell transplantation is attempted as part of post-remission therapy. Stem cell transplants replace stem cells, for example, in patients with relapsed or refractory AML in whom cytogenic or molecular studies predict poorer prognosis with only chemotherapy or targeted therapy. The goal is to destroy all of the cancer cells in the marrow, blood, and other parts of the body using high doses of chemotherapy and/or radiation therapy and then allow replacement blood stem cells to create healthy bone marrow. These cells, called hematopoietic stem cells, develop into healthy bone marrow. Hematopoietic stem cells are blood-forming cells found both in the bloodstream and in the bone marrow. The stem cells in autologous transplants come from the patient's own body. The stem cells in allogeneic transplants are from a person other than the patient. An allogeneic transplant requires a matched donor. A stem cell transplant is carried out soon after first or second remission in patients who relapse. Subjects who relapse after a stem cell transplant may be offered other treatments, including a donor lymphocyte infusion (DLI).

Even after completion of post-remission consolidation, there is a risk that AML can still return. Lower strength medications, e.g. azacytidine, decitabine, midostaurin are given on an ongoing basis for several years to reduce the chance of the disease returning, called recurrence.

While a variety of different treatment regimens are attempted, individually or in combination, to improve patient outcomes, there is currently no single standard of care for patients with relapsed or refractory AML.

The probability of relapse and recurrence of AML is extremely high with all modalities of existing treatment. In addition, AML is refractory to various treatments through chemoresistance. Further, AML is an oligoclonal disease, i.e. a predominant clone at initial presentation of the disease may be non-identical to the clone associated with clinical relapse. The method of the present disclosure of providing untransduced gamma delta T cells Vδ1+ at specific doses provides a useful therapy for a disease that is extremely challenging to treat.

In some aspects, the present disclosure provides a method of treating acute myeloid leukemia by administering to a subject in need thereof a therapeutically effective amount of a composition comprising Vδ1+ gamma delta (γδ) T cells, wherein the acute myeloid leukemia is relapsed or refractory, such that one or more symptoms or biomarkers is improved after treatment.

In some embodiments, the subject has previously been treated with chemotherapy. In some embodiments, the subject has received at least two courses of intensive induction chemotherapy.

In some embodiments, the subject has 5% leukemic blasts in bone marrow. In some embodiments, the subject has greater than about 5%, greater than about 10%, greater than about 20%, greater than about 25%, greater than about 30%, greater than about 35%, greater than about 40%, greater than about 45% or greater than about 50% leukemic blasts in bone marrow.

In some embodiments, the subject has 5% leukemic blasts in peripheral blood. In some embodiments, the subject has greater than about 5%, greater than about 10%, greater than about 20%, greater than about 25%, greater than about 30%, greater than about 35%, greater than about 40%, greater than about 45% or greater than about 50% leukemic blasts in peripheral blood.

In some embodiments, the disclosure provides a method of treatment of a subject in need thereof, wherein the subject is a pediatric subject. In some embodiments, the subject is at least 2 years old. In some embodiments, the subject is at least 12 years old.

In some embodiments, the disclosure provides a method of treatment of a subject in need thereof, wherein, the subject is an adult subject. In some embodiments, the subject is at least 18 years old.

Gamma delta (γδ) T Cells

Gamma delta (γδ) T cells are lymphoid cells that undergo maturation in the thymus. Double-negative thymocytes (CD4− CD8−) are differentiated into T cells expressing γδ T cell receptor (TCR). These cells then migrate to peripheral blood (PB) and mucosal tissues, where they function as primary effectors in the response against infections and cancer, prior to responses of the αβ T cell lineage.

According to the Lefranc & Rabbits's system nomenclature, four subtypes of human γδ T cells are defined by the TCR 8 chain, namely, Vδ1, Vδ2, Vδ3 and Vδ5 (LeFranc M P et al. Cell (1986) 45:237-46). Vδ1 and Vδ2 subtypes are the most predominant while Vδ3 cells comprise a major form of the Vδ1-Vδ2-subtype and found in liver, rather than peripheral blood. Vδ5 cells are found in peripheral blood or tissues.

Vδ1+ γδ T cells recognize target cells and mediate anti-tumor activity through the direct lysis of transformed cells. In one aspect, the method of the present disclosure provides GDX012 as a novel allogeneic cell therapy that is enriched for Vδ1+ γδ T cells for the treatment of relapsed or refractory AML. The cryopreserved drug product is manufactured from a healthy donor leukapheresis that has undergone αβ T cell depletion prior to expansion. GDX012 is a viable (greater than 70% live), pure (CD45 of greater than 90%) suspension for IV infusion consisting mainly of Vδ1+ γδ T cells (greater than 60%), expressing a variety of cellular markers that contribute to their function, recognition and targeting of malignant cells. Analysis of the biodistribution of GDX012 in therapeutic murine xenograft models shows that when administered systemically, GDX012 demonstrates homing to the bone marrow and is detected for at least 28 days. This suggests that in addition to high cytotoxic activity against AML blasts, GDX012 also has the capacity to home to and persist in the bone marrow, providing long-term cytotoxic activity in a target tissue.

### Vδ1+ T Cell Enriched Allogeneic Compositions

In some embodiments, the composition comprises less than 5% residual Vδ2+ cells. In some embodiments, the composition comprises less than 0.5% residual Vδ2+ cells. In some embodiments, the composition comprises less than 0.1% residual Vδ2+ cells. In some embodiments, the composition comprises no residual Vδ2+ cells.

In some embodiments, the composition comprises at least about 90% Vδ1+ cells relative to total live cells. In some embodiments, the composition comprises at least about 91%, 92%, 93%, 94%, 95%, 96%, 97%, 98%, 99%, 99.5%, 99.9% Vδ1+ cells relative to total live cells. In some embodiments, the composition comprises 100% Vδ1+ cells relative to total live cells.

In some embodiments, the Vδ1+ T cells comprises at least 50%, at least 60%, at least 70%, at least 80%, at least 90% of Vδ1+ γδ T cells relative to total live cells.

In some embodiments, the composition comprises at least 50% Vδ1+ γδ T cells relative to total live cells.

In some embodiments, the composition comprises at least 70% Vδ1+ γγ T cells relative to total live cells.

In some embodiments, the composition comprises at least 90% Vδ1+ γδ T cells relative to total live cells.

In some embodiments, the composition comprises at least 99% Vδ1+ γδ T cells relative to total live cells.

In some embodiments, the Vδ1+ T cells comprises at least 90%, at least 91%, at least 92%, at least 93%, at least 94%, at least 95%, at least 96%, at least 97%, at least 98%, at least 99% of Vδ1+ γδ T cells relative to total live cells. In some embodiments, the Vδ1+ T cells comprises at least 99.1% to 99.9% of Vδ1+ γδ T cells relative to total live cells. In some embodiments, the Vδ1+ T cells comprises 100% of Vδ1+ γδ T cells relative to total live cells.

In some embodiments, the composition comprises at least about 60% gamma delta T cells relative to total live cells. In some embodiments, the composition comprises at least about 65%, 70%, 75%, 80%, 85%, 90%, 95%, 99% gamma delta T cells relative to total live cells.

In some embodiments, the allogencic composition comprises an ex vivo expanded cell population enriched for Vδ1+ T cells relative to the starting unexpanded cell population.

In some embodiments, the Vδ1+ composition comprises less than 0.1% αβ T cells relative to total live cells. In some embodiments, the Vδ1+ composition comprises less than 0.09%, less than 0.08%, less than 0.07%, less than 0.06%, less than 0.05%, less than 0.04%, less than 0.03%, less than 0.02% or less than 0.01% αβ T cells.

### Methods of Making Vδ2− Vδ1+ T Cell Enriched Allogeneic Compositions

In some aspects, the Vδ1+ enriched allogeneic T cells are obtained using methods known in the art. For example, the Vδ1+ T cells may be obtained using the methods described in WO2016/198480, WO2017/072367 or WO2018/202808, which are herein incorporated by reference. Methods used selectively expand Vδ1+ T cells (in particular, Vδ2− TCRγδ+ T cells) in culture to high yields suitable for clinical use in treating subjects with relapsed or refractory AML. The methods are carried out on a sample, which may also referred to as a “starting sample”. The methods uses either unfractionated samples or samples which have been enriched for TCRγδ+ T cells.

In some aspects, the Vδ1+ T cell compositions are expanded using exogenous growth factors have improved polyclonality compared to FACS-sorted, unexpanded Vδ1+ T cells simply obtained from peripheral blood (i.e. ex vivo Vδ1+ T cells), therefore in one embodiment, the allogeneic composition comprises Vδ1+ T cells obtained using an expansion method, in particular, wherein said expansion method comprises culturing Vô1+ T cells in the presence of exogenous growth factors.

In some aspects, the sample is any sample that contains gamma delta T cells or precursors thereof including, but not limited to, blood, bone marrow, lymphoid tissue, epithelia, thymus, liver, spleen, cancerous tissues, lymph node tissue, infected tissue, fetal tissue and fractions or enriched portions thereof.

In some embodiments, the Vδ1+ T cells are obtained from a blood sample. The sample includes peripheral blood, umbilical cord blood or fractions thereof, including buffy coat cells, leukapheresis products, peripheral blood mononuclear cells (PBMCs) and low density mononuclear cells (LDMCs). In some embodiments, the blood sample is peripheral blood or a fraction thereof. In some embodiments, the sample is human blood or a fraction thereof. The cells may be obtained from a sample of blood using techniques known in the art such as density gradient centrifugation. For example, whole blood may be layered onto an equal volume of FICOLL-HYPAQUE followed by centrifugation at 400×g for 15-30 minutes at room temperature. The interface material will contain low density mononuclear cells which can be collected and washed in culture medium and centrifuged at 200×g for 10 minutes at room temperature. The sample may be fresh or frozen.

In some embodiments, the Vδ1+ T cells are obtained from a human sample.

As described herein, the compositions and methods of the disclosure may be used with allogeneic derived Vδ1+ T cells, i.e. cells derived from a sample obtained from another donor. In some embodiments, the Vδ1+ T cells are obtained from a healthy donor.

In some aspects, prior to culturing the sample or fraction thereof (such as PBMCs), the sample or fraction thereof may be enriched for certain cell types and/or depleted for other cell types. In some embodiments, the sample is enriched for T cells. In some embodiments, the sample is enriched for TCRγδ+ T cells. For example, the sample may be depleted of TCRα+ T cells, non-TCRγδ+ T cells and/or enriched for CD3+ cells. In some embodiments, the sample is first depleted of TCRα+ T cells, and then enriched for CD3+ cells.

In some aspects, the sample may be enriched or depleted of certain cell types using techniques known in the art. In some embodiments, the cells of a particular phenotype may be depleted by culturing the sample or fraction thereof with an antibody cocktail containing antibodies that bind to specific molecules on the cells to be depleted. Preferably, the antibodies in the cocktail are coupled to magnetic microbeads that can be used to magnetically deplete or enrich target cells when these cells are forced to pass through a magnetic column. In some embodiments, the sample is depleted of αβ T cells.

In some aspects the collection of the Vδ1+ T cells may include the physical collection of Vδ1+ T cells from the culture, isolation of the Vδ1+ T cells from other lymphocytes (e.g. αβ T cells, γδ T cells and/or NK cells) or isolation and/or separation of the Vδ1+ T cells from stromal cells (e.g. fibroblasts). In some embodiments, Vδ1+ T cells are collected by mechanical means (e.g. pipetting). In some embodiments, Vδ1+ T cells are collected by means of magnetic separation and/or labelling. In some embodiments, the Vδ1+ T cells are collected by flow cytometric techniques such as FACS. Thus, in certain embodiments, the Vδ1+ T cells are collected by means of specific labelling the Vδ1+ T cells. It will be appreciated that such collection of Vδ1+ T cells may include the physical removal from the culture, transfer to a separate culture vessel or to separate or different culture conditions.

In some aspects, upon isolation from the sample, the Vδ1+ T cells will generally be part of a larger population of lymphocytes containing, for example, αβ T cells, B cells, and natural killer (NK) cells. In some embodiments, 0.1%-10% of the isolated population of lymphocytes are Vδ1+ T cells, e.g. 1-10% of the isolated population of lymphocytes are Vδ1+ γδ T cells. In some embodiments, the percentage of Vδ1+ T cells is measured in proportion of CD45+ cells (leukocyte common antigen). In some embodiments, the isolated population is depleted of other cell types (e.g. depleted of αβ T cells). In some embodiments, the isolated population of CD45+ cells depleted of αβ T cells comprises at least 0.1% Vδ1+ T cells, such as at least 0.5% Vδ1+ T cells. In most cases, the γδ T cell population (e.g. blood-derived γδ T cell population) will include a predominant population of Vδ1 T cells.

In some aspects, once the cells in the sample have been fractionated and enriched, if desired, the cells may be cultured.

In certain embodiments, the disclosure features methods of expanding Vδ1+ T cells. These methods may be carried out in vitro. In some embodiments, the Vδ1+ T cells are expanded from a population of γδ T cells that has been isolated from a sample as described herein.

In some embodiments, the Vδ1+ T cells are obtained from a sample by a method comprising culturing the sample in a medium comprising a T cell mitogen and a growth factor having interleukin-4-like activity, in the absence of a growth factor having interleukin-15-like activity.

In some embodiments, the Vδ1+ T cells are obtained from a sample by a method comprising culturing the sample in a medium comprising a T cell mitogen and a growth factor having interleukin-15-like activity, in the absence of a growth factor having interleukin-4-like activity.

In some embodiments, the Vδ1+ T cells are obtained from a sample by a method comprising: (1) culturing cells in the sample in a first culture medium comprising a T cell mitogen and a growth factor having interleukin-4-like activity; in the absence of a growth factor having interleukin-15-like activity; and (2) culturing the cells obtained in step (1) in a second culture medium comprising a T cell mitogen and a growth factor having interleukin-15-like activity, in the absence of a growth factor having interleukin-4-like activity.

“In the absence of interleukin-15, interleukin-2 and interleukin-7” and in the absence of interleukin-4″ refer not only to the complete absence of these cytokines in the culture medium, but also include the use of such cytokines at concentration levels so low that they cannot produce a measurable response or physiological effect in target cells and thus can be considered absent for practical purposes. It should be apparent to any one skilled in the art that cells cultured in the first culture medium must not receive a functionally relevant stimulus by IL-2, IL-7 and IL-15 or functionally similar growth factors. Additionally, cells in the second culture medium must not receive a functionally relevant stimulus by IL-4 or functionally similar growth factors. In some embodiments, these cytokines must not be present in the cell culture medium at a final concentration higher than 2 ng/ml; more preferably, not higher than 1 ng/ml, more preferably not higher than 0.1 ng/ml. In some embodiments, these cytokines are absent.

In some embodiments, the growth factor having interleukin-15-like activity is either interleukin-15 (IL-15), interleukin-2 (IL-2), or interleukin-7 (IL-7), preferably IL-15. As used herein, “IL-15” refers to native or recombinant IL-15 or a variant thereof that acts as an agonist for one or more IL-15 receptor (IL-15R) subunits (e.g. mutants, muteins, analogues, subunits, receptor complexes, fragments, isoforms, and peptidomimetics thereof). IL-15, like IL-2, is a known T-cell growth factor that can support proliferation of an IL-2-dependent cell line, CTLL-2.

IL-15 can also refer to IL-15 derived from a variety of mammalian species, including, for example, human, simian, bovine, porcine, equine, and murine. An IL-15 “mutein” or “variant”, as referred to herein, is a polypeptide substantially homologous to a sequence of a native mammalian IL-15 but that has an amino acid sequence different from a native mammalian IL-15 polypeptide because of an amino acid deletion, insertion or substitution. Variants may comprise conservatively substituted sequences, meaning that a given amino acid residue is replaced by a residue having similar physiochemical characteristics. Examples of conservative substitutions include substitution of one aliphatic residue for another, such as lie, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-15 variants are also encompassed by the disclosure. Examples of such variants are proteins that result from alternate mRNA splicing events or from proteolytic cleavage of the IL-15 protein, wherein the IL-15 binding property is retained. Alternate splicing of mRNA may yield a truncated but biologically active IL-15 protein. Variations attributable to proteolysis include, for example, differences in the N- or C-termini upon expression in different types of host cells, due to proteolytic removal of one or more terminal amino acids from the IL-15 protein (generally from 1-10 amino acids).

As used herein, “IL-2” refers to native or recombinant IL-2 or a variant thereof that acts as an agonist for one or more IL-2 receptor (IL-2R) subunits (e.g. mutants, muteins, analogues, subunits, receptor complexes, fragments, isoforms, and peptidomimetics thereof). Such agents can support proliferation of an IL-2-dependent cell line, CTLL-2 (33; American Type Culture Collection (ATCC®) TIB 214).

IL-2 can also refer to IL-2 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-2 variants are also encompassed by the disclosure. Examples of such variants are proteins that result from alternate mRNA splicing events or from proteolytic cleavage of the IL-2 protein, wherein the IL-2 binding property is retained. Alternate splicing of mRNA may yield a truncated but biologically active IL-2 protein. Variations attributable to proteolysis include, for example, differences in the N- or C-termini upon expression in different types of host cells, due to proteolytic removal of one or more terminal amino acids from the IL-2 protein (generally from 1-10 amino acids).

As used herein, “IL-7” refers to native or recombinant IL-7 or a variant thereof that acts as an agonist for one or more IL-7 receptor (IL-7R) subunits (e.g. mutants, muteins, analogues, subunits, receptor complexes, fragments, isoforms, and peptidomimetics thereof). Mature human IL-7 occurs as a 152 amino acid sequence (less the signal peptide, consisting of an additional 25 N-terminal amino acids).

IL-7 can also refer to IL-7 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-7 variants are also encompassed by the disclosure. Examples of such variants are proteins that result from alternate mRNA splicing events or from proteolytic cleavage of the IL-7 protein, wherein the IL-7 binding property is retained. Alternate splicing of mRNA may yield a truncated but biologically active IL-7 protein. Variations attributable to proteolysis include, for example, differences in the N- or C-termini upon expression in different types of host cells, due to proteolytic removal of one or more terminal amino acids from the IL-7 protein (generally from 1-10 amino acids).

In one embodiment, the growth factor having interleukin-4-like activity is interleukin-4 (IL-4).

As used herein, “IL-4” refers to native or recombinant IL-4 or a variant thereof that acts as an agonist for one or more IL-4 receptor (IL-4R) subunits (e.g. mutants, muteins, analogues, subunits, receptor complexes, fragments, isoforms, and peptidomimetics thereof). Such agents can support differentiation of naive helper T cells (ThO cells) to Th2 cells. Mature human IL-4 occurs as a 129 amino acid sequence (less the signal peptide, consisting of an additional 24 N-terminal amino acids).

IL-4 can also refer to IL-4 derived from a variety of mammalian species, including, for example, human, simian, bovine, porcine, equine, and murine. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring IL-4 variants are also encompassed by the disclosure. Examples of such variants are proteins that result from alternate mRNA splicing events or from proteolytic cleavage of the IL-4 protein, wherein the IL-4 binding property is retained. Alternate splicing of mRNA may yield a truncated but biologically active IL-4 protein. Variations attributable to proteolysis include, for example, differences in the N- or C-termini upon expression in different types of host cells, due to proteolytic removal of one or more terminal amino acids from the IL-4 protein (generally from 1-10 amino acids).As used herein, “symptoms” include, but are not limited to, the following:

weight loss, fatigue, fever, night sweats, loss of appetite, weakness, feeling cold, feeling dizzy or lightheaded, headaches, pale skin, shortness of breath, bruising, excess bleeding, frequent or severe nosebleeds, bleeding or clotting problems, headache, slurred speech, equilibrium, blurred vision, facial numbness, enlarged lymph nodes, bone or joint pain and any combinations thereof.

In some embodiments, the Vδ1+ T cells are obtained from a sample by a method comprising: (1) culturing cells in the sample in a first culture medium comprising a T cell mitogen and interleukin-4; in the absence of interleukin-15, interleukin-2 and interleukin-7; and (2) culturing the cells obtained in step (1) in a second culture medium comprising a T cell mitogen and interleukin-15, in the absence of interleukin-4.

Methods of obtaining the Vδ1+ T cells from a sample may comprise additional growth factors. Therefore, in some embodiments, the first or second culture medium, or both culture media, further comprise one or more additional growth factors. Said additional growth factors may be selected from: interferon-γ (IFN-g), interleukin-21 (IL-21), interleukin-1b (IL-1 b), and combinations thereof. In some embodiments, the additional growth factor is IFN-g. These growth factors may be added to one or both culture media to further increase the expansion and purity levels of cultured Vδ1+ T cells. Additional growth factors may include IL-6, IL-7, IL-8, IL-9, IL-12, IL-18, IL-33, IGF-1, human platelet lysate (HPL), and stromal cell-derived factor-1 (SDF-1). In some embodiments, such factors are used in the expansion which selectively promote the expansion of Vδ1+ T cells.

T cell mitogens refer to any agent that can stimulate T cells through TCR signalling including, but not limited to, plant lectins such as phytohemagglutinin (PHA) and concanavalin A (ConA) and lectins of non-plant origin, antibodies that activate T cells, and other non-lectin/non-antibody mitogens. Preferred antibody clones include anti-CD3 antibodies such as OKT-3 and UCHT-1 clones, anti-γδ antibodies such as B1 and IMMU510, or anti-Vδ1 antibodies. Within the context of the present disclosure, antibodies are understood to include monoclonal antibodies (mAbs), polyclonal antibodies, antibody fragments (e.g. Fab, and F(ab′)2), single chain antibodies, single chain variable fragments (scFv) and recombinantly produced binding partners. In some embodiments, the antibody is an anti-CD3 monoclonal antibody (mAb). In some embodiments, the antibody is an anti-Vδ1 antibody. Other mitogens include phorbol 12-myristate-13-acetate (TPA) and its related compounds, such as mezerein, or bacterial compounds (e.g. Staphylococcal enterotoxin A (SEA) and Streptococcal protein A). The T cell mitogens may be soluble or immobilized and more than one T cell mitogen may be used in the method.

In some embodiments, the T cell mitogen is an antibody or a fragment thereof. The antibody or fragment thereof may be an anti-CD3 antibody, for example OKT-3. Alternatively, or additionally, the antibody or fragment thereof may be an anti-TCRγδ antibody, such as a pan-γδ TCR antibody or an anti-TCRδ1 antibody. References herein to “culturing” include the addition of cells to a media comprising growth factors and/or essential nutrients required and/or preferred by the cells and/or non-haematopoietic tissue sample. Culturing may be by selective expansion, such as by choosing culturing conditions where Vδ1+ T cells are preferentially expanded over other cells types present in the sample. Alternatively, the expansion conditions are not selective and culturing may be followed by depletion of non-target cells (e.g. cells other than Vδ1+ T cells, such as αβ T cells). Alternatively, the expansion conditions are not selective and depletion of non-target cells (e.g. cells other than Vδ1+ T cells, such as αβ T cells) occurs prior to culturing.

In some embodiments, the culturing is performed in the absence of feeder cells.

In some embodiments, the culturing is performed in the absence of substantial stromal cell contact. In some embodiments, the culturing is performed in the absence of substantial fibroblast cell contact.

In some embodiments, the Vδ1+ T cells are collected after at least 11 days of culturing, such as at least 14 days of culturing. In some embodiments, the duration of culture according to the methods defined herein is at least 14 days. In some embodiments, the duration of culture according to the methods defined herein is less than 45 days, such as less than 30 days, such as less than 25 days. In some embodiments, the duration of culture according to the methods defined herein is between 14 days and 35 days, such as between 14 days and 21 days. In some embodiments, the duration of culture according to the methods defined herein is about 21 days.

In some embodiments, the culturing is performed for a duration (e.g. at least 5 days, at least 6 days, at least 7 days, at least 8 days, at least 9 days, at least 10 days, at least 11 days, at least 12 days, at least 13 days, at least 14 days, at least 21 days, at least 28 days, or longer, e.g. from 5 days to 40 days, from 7 days to 35 days, from 14 days to 28 days, or about 21 days) in an amount effective to produce an expanded population of Vδ1+ T cells. In some embodiments, the culturing is for a period of several hours (e.g. about 2, 3, 4, 5, 6, 7, 8, 9, 10, 15, 18, or 21 hours) to about 35 days (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days). In some embodiments, the culturing is for a period of 14 to 21 days.

It will be understood that if two culture media are used, the culturing in each media may occur for different lengths of time. For example, cells are cultured in the first culture medium for a period of time ranging from about 2 days to about 21 days. In some embodiments, cells are cultured from about 3 days to about 14 days. In some embodiments, cells are cultured from about 4 days to 8 days. The cells may be cultured in the second culture medium for a period of time ranging from about 2 days to about 30 days. In some embodiments, cells are cultured from about 5 days to about 21 days. In some embodiments, cells are cultured from about 10 days to 15 days.

In some embodiments, the culturing is performed in a vessel comprising a gas permeable material. Such materials are permeable to gases such as oxygen, carbon dioxide and/or nitrogen to allow gaseous exchange between the contents of the vessel and the surrounding atmosphere. It will be appreciated that references herein to “vessel” include culture dishes, culture plates, single-well dishes, multi-well dishes, multi-well plates, flasks, multi-layer flasks, bottles (such as roller bottles), bioreactors, bags, tubes and the like. Such vessels are known in the art for use in methods involving expansion of non-adherent cells and other lymphocytes. Vessels comprising a gas permeable material have been found to increase the yield of isolated Vδ1+ T cells. Such vessels were also found to preferentially support Vδ1+ T cells and other lymphocytes over fibroblasts and other stromal cells (e.g. epithelial cells), including adherent cell-types. In some embodiments, fibroblasts and/or other stromal cells (e.g. epithelial cells) are absent from cultures performed in vessels comprising a gas permeable material.

Such vessels comprising gas permeable materials may additionally comprise a gas permeable material that is non-porous. Thus, in some embodiments, the gas permeable material in non-porous. In some embodiments, the gas permeable material is a membrane film such as silicone, fluoroethylene polypropylene, polyolefin, or ethylene vinyl acetate copolymer. Furthermore, such vessels may comprise only a portion of gas permeable material, gas permeable membrane film or non-porous gas permeable material. Thus, in some embodiments, the vessel includes a top, a bottom and at least one sidewall, wherein at least part of the said vessel bottom comprises a gas permeable material that is in a substantially horizontal plane when said top is above said bottom. In some embodiments, the vessel includes a top, a bottom, and at least one sidewall, wherein at least a part of said bottom comprises the gas permeable material that is in a horizontal plane when said top is above said bottom. In some embodiments, the vessel includes a top, a bottom and at least one sidewall, wherein the said at least one sidewall comprises a gas permeable material which may be in a vertical plane when said top is above said bottom, or may be a horizonal plane when said top is not above said bottom. It will be appreciated that in such embodiments, only a portion of said bottom or said side wall may comprise a gas permeable material. Alternatively, the entire of said bottom or entire of said sidewall may comprise a gas permeable material. In some embodiments, said top of said vessel comprising a gas permeable material may be sealed, for example by utilization of an O-ring. Such embodiments will be appreciated to prevent spillage or reduce evaporation of the vessel contents. In some embodiments, the vessel comprises a liquid sealed container comprising a gas permeable material to allow gas exchange. In some embodiments, said top of said vessel comprising a gas permeable material is in the horizonal plane and above said bottom and is not sealed. In some embodiments, said top is configured to allow gas exchange from the top of the vessel. In some embodiments, said bottom of the gas permeable container is configured to allow gas exchange from the bottom of the vessel. In some embodiments, said vessel comprising a gas permeable material may be a liquid sealed container and further comprise inlet and outlet ports or tubes. In some embodiments, the vessel comprising a gas permeable material includes a top, a bottom and optionally at least one sidewall, wherein at least a part of said top and said bottom comprise a gas permeable material and, if present, at least part of the at least one sidewall comprises a gas permeable material. Example vessels are described in WO2005/035728 and U.S. Pat. No. 9,255,243 which are herein incorporated by reference. These vessels are also commercially available, such as the G-REX® cell culture devices provided by Wilson Wolf Manufacturing, such as the G-REX6 well-plate, G-REX24 well-plate and the G-REX10 vessel.

In some embodiments, the sample is cultured in media which is substantially free of serum (e.g. serum-free media or media containing a serum-replacement (SR)). Thus, in some embodiments, the sample is cultured in serum-free media. Such serum free medium may also include serum replacement medium, where the serum replacement is based on chemically defined components to avoid the use of human or animal derived serum. In some embodiments, the sample is cultured in media which contains serum (e.g. human AB serum or fetal bovine serum (FBS)). In some embodiments, the sample is cultured in media which contains serum-replacement. In some embodiments, the sample is cultured in media which contains no animal-derived products. Culturing the sample in serum-free media has the advantage of avoiding issues with filtration, precipitation, contamination and supply of serum. Furthermore, animal derived products are not favoured for use in clinical grade manufacturing of human therapeutics.

Numerous basal culture media suitable for use in the proliferation of γδ T cells are available, in particular medium, such as AIM-V, Iscoves medium and RPMI-1640 (Life Technologies). The medium may be supplemented with other media factors as defined herein, such as serum, serum proteins and selective agents, such as antibiotics. For example, in some embodiments, RPMI-1640 medium containing 2 mM glutamine, 10% FBS, 10 mM HEPES, pH 7.2, 1% penicillin-streptomycin, sodium pyruvate (1 mM; Life Technologies), non-essential amino acids (e.g., 100 mM Gly, Ala, Asn, Asp, Glu, Pro and Ser; 1X MEM non-essential amino acids (Life Technologies)), and 10 pl/L β-mercaptoethanol. In an alternative embodiment, AIM-V medium may be supplemented with CTS Immune serum replacement and amphotericin B. Conveniently, cells are cultured at 37° C. in a humidified atmosphere containing 5% C0₂ in a suitable culture medium during isolation and/or expansion. Examples of other ingredients that may be added to the culture media, include, but are not limited to, plasma or serum, purified proteins such as albumin, a lipid source such as low density lipoprotein (LDL), vitamins, amino acids, steroids and any other supplements supporting or promoting cell growth and/or survival.

In some aspects, the Vδ1+ T cells obtained according to the described methods can be separated from other cells that may be present in the final culture using techniques known in the art including fluorescence activated cell sorting, immunomagnetic separation, affinity column chromatography, density gradient centrifugation and cellular panning.

In some aspects, the obtained Vδ1+ T cells (GDX012) may be immediately used in the therapeutic, experimental or commercial applications described herein or the cells may be cryopreserved for use at a later date.

### Methods of Treatment of Relapsed or Refractory AML

In some aspects of the present disclosure, provided herein is a method of treating acute myeloid leukemia by administering to a subject in need thereof a therapeutically effective amount of a composition comprising Vδ1+ gamma delta (γδ) T cells, wherein the acute myeloid leukemia is relapsed or refractory, such that one or more symptoms or biomarkers is improved after treatment. The allogeneic composition may comprise a dose that is suitable for administration to a patient. In some embodiments, provided herein is a dose of an allogeneic composition comprising γ81+ T cells for use in the treatment of a patient with relapsed or refractory AML.

In some aspects, the Vδ1+ gamma delta (γδ) T cells are untransduced. In some embodiments, the Vδ1+ gamma delta (γδ) T cells do not express a chimeric antigen receptor (CAR). In some embodiments, the Vδ1+ gamma delta (γδ) T cells express a chimeric antigen receptor (CAR).

In some embodiments, the therapeutically effective amount of Vδ1+ gamma delta (γδ) T cells comprises about 8×10¹⁰, 4×10¹⁰, 8×10⁹, 4×10⁹, 2.4×10⁹, 1.2×10⁹, 8×10⁸, 4×10⁸, 8×10⁷ or 4×10⁷ live T cells.

In some embodiments, the therapeutically effective amount of Vδ1+ gamma delta (γδ) T cells comprises about 8×10¹⁰ live T cells. In some embodiments, the therapeutically effective amount of Vδ1+ gamma delta (γδ) T cells comprises about 4×10¹⁰ live T cells. In some embodiments, the therapeutically effective amount of Vδ1+ gamma delta (γδ) T cells comprises about 8×10⁹ live T cells. In some embodiments, the therapeutically effective amount of Vδ1+ gamma delta (γδ) T cells comprises about 4×10⁹ live T cells. In some embodiments, the therapeutically effective amount of Vδ1+ gamma delta (γδ) T cells comprises about 2.4×10⁹ live T cells. In some embodiments, the therapeutically effective amount of Vδ1+ gamma delta (γδ) T cells comprises about 1.2×10⁹ live T cells. In some embodiments, the therapeutically effective amount of Vδ1+ gamma delta (γδ) T cells comprises about 8×10⁸ live T cells. In some embodiments, the therapeutically effective amount of Vδ1+ gamma delta (γδ) T cells comprises about 4×10⁸ live T cells. In some embodiments, the therapeutically effective amount of Vδ1+ gamma delta (γδ) T cells comprises about live 8×10⁷ T cells. In some embodiments, the therapeutically effective amount of Vδ1+ gamma delta (γδ) T cells comprises about 4×10⁷ live T cells. In some embodiments, the therapeutically effective amount of Vδ1+gamma delta (γδ) T cells comprises less than about 4×10⁷ live T cells.

In some embodiments, the therapeutically effective amount comprises less than about 5×10⁴ alpha beta T cells. In some embodiments, the therapeutically effective amount comprises less than about 1×10⁴ alpha beta T cells.

In some embodiments, the composition may comprise a dose (such as a therapeutically effective dose) for administration to a patient. In some embodiments, the patient is administered a dose of Vδ1+T cells calculated per kg body weight of the patient. In some embodiments, a dose of Vδ1+T cells as described herein comprises about 1×10⁵, 5×10⁵, 1×10⁶, 1.5×10⁶, 2×10⁶, 3×10⁶, 5×10⁶, 1×10⁷, 1.5×10⁷, 2×10⁷, 3×10⁷, 5×10⁷, 1×10⁸, 2×10⁸, or 5×10⁸ cells/kg. In some embodiments, a dose of Vδ1+T cells comprises at least about 1×10⁵, 5×10⁵, 1×10⁶, 1.5×10⁶, 2×10⁶, 3×10⁶, 5×10⁶, 1×10⁷, 1.5×10⁷, 2×10⁷, 3×10⁷, 5×10⁷, 1×10⁵, 2×10⁸, or 5×10⁸ cells/kg. In some embodiments, a dose of Vδ1+ T cells comprises up to about 1×10⁶, 1.5×10⁶, 2×10⁶, 3×10⁶, 5×10⁶, 1×10⁷, 1.5×10⁷, 2×10⁷, 3×10⁷, 5×10⁷, 1×10⁸, 2×10⁵, or 5×10⁸ cells/kg. In some embodiments, a dose of Vδ1+ T cells comprises about 1×10⁶-1×10⁸ cells/kg.

In some embodiments, the dose of the allogeneic composition may comprise no more than 5×10⁴ab T cells/kg, such as no more than about 10⁴, 10³ or 10² αβ T cells/kg. Therefore, in some embodiments the dose comprises less than about 5×10⁴ ab T cells/kg. In some embodiments, the dose comprises less than about 1×10⁴ αβ T cells/kg.

In some embodiments, the allogeneic composition is frozen and then thawed before administration. In some embodiments, the dose of the allogeneic composition is calculated prior to freezing. In some embodiments, the dose is calculated after thawing. In some embodiments, the allogeneic composition is not frozen. In some embodiments, the method additionally comprises administration of chemotherapy. In some embodiments, the patient is treated with chemotherapy at least 3 days prior to administration of the allogeneic composition. In some embodiments, the chemotherapy is fludarabine and cyclophosphamide.

In some embodiments, the subject receives an initial administration of Vδ1+ T cells (e.g. an initial administration of 10⁶ to 10⁸ Vδ1+ T cells per kg body weight of the subject, e.g. 10⁶ to 10⁷ Vδ1+ T cells per kg body weight of the subject), and one or more (e.g. 2, 3, 4, or 5) subsequent administrations of Vδ1+ T cells. In some embodiments, the one or more subsequent administrations are administered 30 days after the previous administration. In some embodiments, the one or more subsequent administrations are administered 15 days after the previous administration. In some embodiments, the one or more subsequent administrations are administered less than 15 days, e.g., 14, 13, 12, 11, 10, 9, 8, 7, 6, 5, 4, 3, or 2 days after the previous administration, e.g., less than 4, 3, or 2 days after the previous administration.

In some embodiments, one or more additional therapeutic agents are administered to the subject. The additional therapeutic agent may be selected from the group consisting of an immunotherapeutic agent, a cytotoxic agent, a growth inhibitory agent, a radiation therapy agent, an anti-angiogenic agent, or a combination of two or more agents thereof. The additional therapeutic agent is administered concurrently with, prior to, or after administration of the expanded Vδ1+ T cells. The additional therapeutic agent is an immunotherapeutic agent, which may act on a target within the subject's body (e.g., the subject's own immune system) and/or on the transferred Vδ1+ T cells.

In some embodiments, the administration of the compositions is carried out by any convenient route, for example, the compositions described herein may be administered to a patient intravenously, transarterially, subcutaneously, intradermally, intratumorally, intranodally, intramedullary, intramuscularly, by intravenous injection, or intraperitoneally, e.g., by intradermal or subcutaneous injection. In some embodiments, the compositions are administered as an intravenous infusion.

In some embodiments, the administration of the composition is carried out in one or more doses. In some embodiments, the composition is administered in one dose. In some embodiments, the composition is administered once a week, once every two weeks, once a month or once in two months. In some embodiments, the composition is administered once a month. In some embodiments, the composition is administered for a period of 1 month, 2 months, 4 months, 6 months, 12 months, 14 months, 18 months or 24 months. In some embodiments, the composition is administered for a period of greater than 24 months.

### Lymphodepletion Therapy

According to the present disclosure, in some embodiments, administration of Vδ1+ γδ T cells is preceded by de-intensified lymphodepleting chemotherapy.

In some embodiments, the lymphodepleting therapy comprises administration of cytarabine and fludarabine.

In some embodiments, the lymphodepletion therapy comprises at least one dose of cytarabine and at least one dose of fludarabine.

In some embodiments, a subject is treated with lymphodepletion at least one time prior to administration of GDX012. In some embodiments, lymphodepletion is performed before one or more additional doses of GDX012 is administered. In some embodiments, lymphodepletion is followed by at least two doses of GDX012 therapy, with the two doses separated by a time interval.

In some embodiments, lymphodepletion is performed prior to the inception of each treatment cycle of GDX012, if multiple treatment cycles are required (e.g., the subject requires further treatment). For example, in several embodiments, a subject undergoes lymphodepletion, receives a plurality of doses of GDX012 cells according to a treatment regimen, and if necessary, undergoes a second lymphodepletion followed by a second treatment regimen.

In some embodiments, the lymphodepletion chemotherapy comprises administration of mitoxantrone, etoposide, cytarabine or anthracycline. In some embodiments, the lymphodepletion chemotherapy comprises administration of 7 days of cytarabine and 3 days of anthracycline. In some embodiments, cytarabine is administered with venetoclax or other B-cell lymphoma 2 (BCL2) inhibitors. In some embodiments, lymphodepletion chemotherapy comprises administration of at least two cycles of hypomethylating agent. In some embodiments, lymphodepletion chemotherapy comprises administration of at least three cycles of hypomethylating agent. In some embodiments, lymphodepletion chemotherapy comprises at least 4 cycles of monotherapy with hypomethylating agents.

In some embodiments, treatment by the method of the present disclosure leads to improvement of one or more symptoms or biomarkers of relapsed or refractory acute myeloid leukemia, for example, bone marrow blasts are substantially absent or reduced to less than 5%. In some embodiments, bone marrow blasts are less than 5%. In some embodiments, bone marrow blasts are from between 5% to 25%. In some embodiments, bone marrow blasts are less than 10%. In some embodiments, bone marrow blasts are less than 15%. In some embodiments, bone marrow blasts are less than 20%. In some embodiments, bone marrow blasts are less than 25%.

In some aspects, treatment leads to improvement of one or more symptoms or biomarkers of relapsed or refractory acute myeloid leukemia, for example, peripheral blood blasts are substantially absent. In some embodiments, peripheral blood blasts are less than 5%. In some embodiments, peripheral blood blasts are from between 5% to 25%. In some embodiments, peripheral blood blasts are less than 10%. In some embodiments, peripheral blood blasts are less than 15%. In some embodiments, peripheral blood blasts are less than 20%. In some embodiments, peripheral blood blasts are less than 25%.

In some embodiments, one or more symptoms or biomarkers that is improved after treatment is decrease of pretreatment bone marrow blast percentage by at least 50% based on European Leukemia Net (ELN) 2022 response criteria for AML.

In some embodiments, a further symptom or biomarker that is improved upon treatment is the absence of extramedullary disease. Haemotological parameters are improved such as an absolute neutrophil count (ANC) of 0.5×10⁹/Liters or greater, 1×10⁹/Liters or greater; a platelet count of 50×10⁹/Liters or greater, 100×10⁹/Liters or greater.

In some embodiments, the measurable residual disease after treatment is less than 0.1%. In some embodiments, MRD is measured by flow cytometry of bone marrow cells and/or blood.

In some embodiments, the γδ T cells express CD27. For example, the Vδ1+ T cells may have a frequency of CD27+ cells of greater than 10%, greater than 20%, greater than 30%, greater than 40%, greater than 50%, greater than 60%, greater than 70%, greater than 80% or greater than 90%. Alternatively, the γδ T cells may have a frequency of CD27+ cells of about 10%, about 20%, about 30%, about 40%, about 50%, about 60%, about 70%, about 80% or about 90%. In certain embodiments, the Vδ1+ T cells have a frequency of CD27+ cells of greater than 10%. Thus, in one embodiment, the Vδ1+ T cells have a frequency of CD27+ cells of about 20%. In a further embodiment, the Vδ1+ T cells have a frequency of CD27+ cells greater than 20%. In one embodiment, the Vδ1+ T cells have a frequency of CD27+ cells of about 20%.

In some embodiments, the Vδ1+ T cells have a low proportion of cells expressing TIGIT, an immune checkpoint receptor on cytotoxic, memory, and Tregs, as well as NK cells. For example, the Vδ1+ T cells may have a frequency of TIGIT+ cells of less than 90%, less than 80%, less than 70%, less than 60%, less than 50%, less than 40%, less than 30%, less than 20% or less than 10%. Alternatively, the Vδ1+ T cells may have a frequency of TIGIT+ cells of about 90%, about 80%, about 70%, about 60%, about 50%, about 40%, about 30%, about 20% or about 10%. In some embodiments, the Vδ1+ T cells do not substantially express TIGIT. In some embodiments, the Vδ1+ T cells express CD27 and/or do not substantially express TIGIT.

Pharmaceutical compositions may include expanded Vδ1+ T cell compositions as described herein in combination with one or more pharmaceutically or physiologically acceptable carrier, diluents, or excipients. Such compositions may include buffers such as neutral buffered saline or phosphate buffered saline; carbohydrates such as glucose, mannose, sucrose or dextrans, mannitol; proteins; polypeptides or amino acids such as glycine; antioxidants; chelating agents such as EDTA or glutathione; adjuvants (e.g. aluminium hydroxide); and preservatives. Cryopreservation solutions which may be used in the pharmaceutical compositions of the disclosure include, for example, DMSO. Compositions can be formulated for any suitable administration, e.g. for intravenous administration.

In some embodiments, the pharmaceutical composition is substantially free of, e.g. there are no detectable levels of a contaminant, e.g. of endotoxin or mycoplasma.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

Various aspects of the invention are described in further detail in the following examples. The following examples describe some of the preferred modes of making and practicing the present invention. However, it should be understood that these examples are for illustrative purposes only and are not meant to limit the scope of the invention.

### Example 1. A Clinical Study to Assess Safety and Efficacy of GDX012 in Patients with Relapsed or Refractory Acute Myeloid Leukemia

This example illustrates a Phase 1/2a, open-Label, dose escalation, and dose expansion study that will be conducted to assess the safety and efficacy of GDX012 in patients with relapsed or refractory Acute Myeloid Leukemia.

The multi-center trial will enroll approximately 53 patients in two phases, dose escalation and dose expansion. During Phase 1 (sequential dose escalation), participants will be assigned to one of the following treatment groups each consisting of approximately 3 to 6 participants to receive GDX012 at one of the three dose levels for a period of 4 to 6 months.

One group of participants will receive GDX012 Dose 1 (weight-based), of between about 4×10⁸ to about 8×10⁸ cells by intravenous (IV) infusion on Day 1 of Phase 1 after lymphodepleting chemotherapy. Some patients may be eligible for a second dose. For example, participants who achieve a response based on European Leukemia Net (ELN) 2022 response criteria from the initial GDX012 infusion at Day 30, do not experience a dose limiting toxicity (DLT), and are healthy optionally receive a second dose of GDX012 Dose 1 of between about 4×10⁸ to about 8×10⁸ cells by intravenous infusion preceded by a de-intensified lymphodepleting chemotherapy. Exemplary lymphodepletion includes treatment with fludarabine and cytarabine. Chemotherapeutic agents, fludarabine and cyclophosphamide can possibly be administered as per standard of care.

Another group of participants will receive GDX012 Dose 2 (weight-based), of between about 1.2×10⁹ to about 2.4×10⁹ cells by IV infusion on Day 1 of Phase 1 after lymphodepleting chemotherapy. Some patients may be eligible for a second dose. For example, participants who achieve a response based on ELN 2022 response criteria from the initial GDX012 infusion at Day 30, do not experience a DLT, and are healthy optionally receive a second dose of GDX012 Dose 2 of between about 1.2×10⁹ to about 2.4×10⁹ cells preceded by a de-intensified lymphodepleting chemotherapy. Exemplary lymphodepletion is achieved by treatment with fludarabine and cytarabine. Chemotherapeutic agents, fludarabine and cyclophosphamide can possibly be administered as per standard of care.

A third group of participants will receive GDX012 Dose 3 (weight-based) of between about 4×10⁹ to about 8×10⁹ cells by IV infusion on Day 1 of Phase 1 after lymphodepleting chemotherapy. Some patients may be eligible for a second dose. For example, participants who achieve a response based on ELN 2022 response criteria from the initial GDX012 infusion at Day 30, do not experience a DLT, and are healthy optionally receive a second dose of GDX012 Dose 3 of between about 4×10⁹ to about 8×10⁹ cells preceded by a de-intensified lymphodepleting chemotherapy. Exemplary lymphodepletion is achieved by treatment with fludarabine and cytarabine. Chemotherapeutic agents, fludarabine and cyclophosphamide can possibly be administered as per standard of care.

From the results of the Phase 1 study, one or two dose levels will be selected for Phase 2a. The recommended phase 2 dose (RP2D) will be determined and depending on, for example, safety, tolerability, efficacy, and cellular kinetics (CK) from the dose escalation phase, response-evaluable participants will be enrolled in Phase 2a (dose expansion) of the study based on whether a single dose level or two dose levels are selected from Phase 1. Accordingly, participants will be enrolled in Phase 2a to receive GDX012 at RP2D (weight-based) as IV infusion on Day 1 in Phase 2.

The following will be primary outcome measures that will be recorded:
a. Number of participants with dose limiting toxicities (DLTs);
b. Maximum tolerated dose (MTD) of GDX012;
c. Number of participants with adverse events, wherein an adverse event (AE) can be defined as any untoward medical occurrence in a clinical investigation participant administered a drug; wherein this is necessarily a causal relationship with this treatment. An AE can therefore be any unfavorable and unintended sign, symptom, or disease temporally associated with the use of a drug, whether or not it is considered related to the drug.

As a secondary outcome measure, disease response includes participants achieving:
(a) Complete response [CR], characterized, for example, by bone marrow blasts of less than 5%; absence of circulating blasts; absence of extramedullary disease; absolute neutrophil count (ANC) of at least 1.0×10⁹/Liters; platelet count of at least 100×10⁹/Liters;
(b) Complete response with incomplete hematologic recovery [CRi], characterized by all CR criteria except for residual neutropenia of less than 1.0×10⁹/Liters or thrombocytopenia of less than 100×10⁹/Liters;
(c) Complete response with partial hematologic recovery [CRh], characterized by ANC of at least 0.5×10⁹/Liters and platelet count of at least 50×10⁹/Liters, wherein otherwise all other CR criteria are met;
(d) Morphological leukemia-free state [MLFS], characterized by bone marrow blasts of less than 5%; absence of blasts; absence of extramedullary disease; and no hematologic recovery required; or
(c) Partial response [PR], characterized by all hematologic criteria of CR; decrease of bone marrow blast percentage to 5% to 25%; and decrease of pretreatment bone marrow blast percentage by at least 50% based on 2022 ELN response criteria for AML.
   The results of the study will demonstrate for example, safety and tolerability, of GDX012 in subjects with relapsed or refractory Acute Myeloid Leukemia. The results will also show how treatment with GDX012 is tolerated and determine the optimal dose of GDX012 for therapy. The study will also demonstrate disease response, i.e., an additional aim of the study will determine if AML is reduced or absent after treatment with GDX012.

### Example 2. Lymphodepletion Treatment in Subjects with Relapsed or Refractory Acute Myeloid Leukemia Prior to GDX012 Therapy

Subjects will receive lymphodepletion therapy prior to treatment with GDX012. Lymphodepletion is a conditioning pre-treatment carried out to reduce the population of circulating lymphocytes, prior to GDX012 therapy. In this example, cytarabine and fludarabine (Fludara) are administered. The lymphodepletion treatment comprises administering to a subject one or more doses of cytarabine and one or more doses of fludarabine prior to GDX012 therapy.

A dosing regimen will be designed to evaluate three doses of Vδ1+ cells that are administered to subjects suffering from relapsed or refractory acute myeloid leukemia. The dosing cycle will be preceded by a conditioning phase during which a subject undergoes lymphodepletion using cytarabine and fludarabine. At day 1, the subject will receive the first of one of three different doses (weight based) of GDX012 by IV infusion on Day 1 of Phase 1 after lymphodepleting chemotherapy as described in Example 1: Dose 1, of between about 4×10⁸ to about 8×10⁸ cells, Dose 2, of between about 1.2×10⁹ to about 2.4×10⁹ cells and Dose 3, of between about 4×10⁹ to about 8×10⁹ cells.

Some subjects will receive a second treatment at the same dose as the first, after a second round of lymphodepletion therapy. For example, some patients who achieve a response based on ELN 2022 response criteria from the initial GDX012 infusion at Day 30, do not experience a DLT, and are healthy, are treated with a second round of cytarabine and fludarabine and subsequently receive the second of one of three different doses (weight based) of GDX012 by IV infusion on Day 1 of Phase 1 after lymphodepleting chemotherapy as described in Example 1: Dose 1, of between about 4×10⁸ to about 8×10⁸ cells, Dose 2, of between about 1.2×10⁹ to about 2.4×10⁹ cells and Dose 3, of between about 4×10⁹ to about 8×10⁹ cells.

Primary outcome measures include: (1) incidence, nature, and severity of treatment related adverse events will be evaluated. An adverse event is any unfavorable and unintended sign including clinically significant abnormal laboratory findings, symptom or disease. This is to be measured 30 days after last dose of the Vδ1+ cells and (2) proportion of subjects experiencing dose-limiting toxicities (DLTs) of the Vδ1+ cells, with DLTs defined as adverse events attributable to treatment that occur during Cycle 1 and meet protocol specified criteria. This is to be measured 28 days from first dose of Vδ1+ cells.

Secondary outcome measures include: (1) assessment of Vδ1+ cell half-life, measured as the time required for 50% reduction from maximum amount of circulating Vδ1+ cells. This is to be measured 28 days from first dose of Vδ1+ cells; (2) Vδ1+ cell duration of persistence by measuring amount of Vδ1+ cells in peripheral blood every 3 months after dosing to determine persistence. This will be measured for up to 2 years after last dose of Vδ1+ cells; (3) evaluation of host immune response against Vδ1+ cells through serum samples that will be measured for antibodies against the Vδ1+ cells. This will be measured for up to 2 years after last dose of Vδ1+ cells; (4) objective response rate to Vδ1+ cells by measuring the percentage of subjects with complete and partial response. AML subjects will be assessed for anti-tumor activity of Vδ1+ cells based on the updated ELN criteria.

Results from this study will show that lymphodepletion with cytarabine and fludarabine reduces circulating lymphocytes and conditions the subject for subsequent treatment with GDX012.

Results from this study will show that administration of GDX012 in one of three doses (either administered once or twice at each dose) after lymphodepletion treatment with cytarabine and fludarabine is favorably tolerated and show limited adverse events, and limited DLTs. Results from this study will demonstrate that Vδ1+ cells show an extended half-life as well as enhanced duration of persistence, and induce limited host immune response and a clinically meaningful objective response rate (e.g., reductions in tumor burden).

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of examples only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method of treating acute myeloid leukemia by administering to a subject in need thereof a therapeutically effective amount of an allogeneic composition comprising Vδ1+gamma delta (γδ) T cells, wherein the acute myeloid leukemia is relapsed or refractory.

2. The method of claim 1, wherein one or more symptoms or biomarkers is improved after the administration.

3. The method of claim 1 or 2, wherein the gamma delta T cells are untransduced.

4. The method of any one of the preceding claims, wherein the composition comprises at least about 65%, 70%, 75%, 80%, 85%, 90%, 95%, 99% gamma delta T cells relative to total live cells.

5. The method of any one of claims 1-3, wherein the composition comprises at least 50% Vδ1+ γδ T cells (e.g., at least 60%) relative to total live cells.

6. The method of claim 4, wherein the composition comprises at least 70% Vδ1+ γδ T cells relative to total live cells.

7. The method of claim 5, wherein the composition comprises at least 90% Vδ1+ γδ T cells relative to total live cells.

8. The method of claim 7, wherein the composition comprises at least 99% Vδ1+ γδ T cells relative to total live cells.

9. The method of claim 1 or 2, wherein the composition comprises less than 5% residual Vδ2+ cells.

10. The method of claim 9, wherein the composition comprises less than 0.5% residual Vδ2+ cells.

11. The method of claim 10, wherein the composition comprises less than 0.1% residual Vδ2+ cells.

12. The method of any one of the preceding claims, wherein the subject has previously been treated with chemotherapy.

13. The method of any one of the preceding claims, wherein the subject has received at least two courses of intensive induction chemotherapy.

14. The method of any one of the preceding claims, wherein the subject has 5% or greater than about 5% leukemic blasts in bone marrow.

15. The method of any one of the preceding claims, wherein the subject has 5% or greater than about 5% leukemic blasts in peripheral blood.

16. The method of any one of the preceding claims, wherein the therapeutically effective amount comprises about 8×10¹⁰, 4×10¹⁰, 8×10⁹, 4×10⁹, 2.4×10⁹, 1.2×10⁹, 8×10⁸, 4×10⁸, 8×10⁷ or 4×10⁷ live T cells.

17. The method of claim 16, wherein the therapeutically effective amount comprises about 8×10⁹ live T cells.

18. The method of claim 16, wherein the therapeutically effective amount comprises about 4×10⁹ live T cells.

19. The method of claim 16, wherein the therapeutically effective amount comprises about 2.4×10⁹ live T cells.

20. The method of claim 16, wherein the therapeutically effective amount comprises about 1.2×10⁹ live T cells.

21. The method of claim 16, wherein the therapeutically effective amount comprises about 8×10⁸ live T cells.

22. The method of claim 16, wherein the therapeutically effective amount comprises about 4×10⁸ live T cells.

23. The method of claim 16, wherein the therapeutically effective amount comprises less than about 4×10⁸ live T cells.

24. The method of claim 16, wherein the therapeutically effective amount comprises less than about 5×10⁴ alpha beta T cells/kg.

25. The method of claim 16, wherein the therapeutically effective amount comprises less than about 1×10⁴ alpha beta T cells/kg.

26. The method of any one of the preceding claims, wherein the gamma delta T cells do not express a chimeric antigen receptor (CAR).

27. The method of any one of the preceding claims, wherein the gamma delta T cells express a chimeric antigen receptor (CAR).

28. The method of any one of the preceding claims, wherein the composition is administered intravenously, transarterially, subcutaneously, intradermally, intratumorally, intranodally, intramedullary, intramuscularly, or intraperitoneally.

29. The method of any one of the preceding claims, wherein the composition is administered intravenously.

30. The method of any one of the preceding claims, wherein the composition is administered in one or more doses.

31. The method of claim 30, wherein the composition is administered in one dose.

32. The method of any one of the preceding claims, wherein the composition is administered once a week, once every two weeks, once a month or once in two months.

33. The method of any one of the preceding claims, wherein the composition is administered once a month.

34. The method of any one of the preceding claims, wherein the composition is administered for a period of 1 month, 2 months, 4 months, 6 months, 12 months, 14 months, 18 months or 24 months.

35. The method of any one of the preceding claims, wherein the composition is administered for a period of greater than 24 months.

36. The method of any one of the preceding claims, wherein the chemotherapy comprises administration of mitoxantrone, etoposide, cytarabine or anthracycline.

37. The method of any one of the preceding claims, wherein the chemotherapy comprises administration of 7 days of cytarabine and 3 days of anthracycline.

38. The method of any one of the preceding claims, wherein the cytarabine is administered with venetoclax, fludarabine or other B-cell lymphoma 2 (BCL2) inhibitors.

39. The method of any one of the preceding claims, wherein chemotherapy comprises administration of at least two cycles of hypomethylating agent.

40. The method of any one of the preceding claims, wherein the chemotherapy comprises at least 4 cycles of monotherapy with hypomethylating agents.

41. The method of any one of the preceding claims, wherein the subject is at least 2 years old.

42. The method of any one of the preceding claims, wherein the subject is at least 12 years old.

43. The method of any one of the preceding claims, wherein the subject is at least 18 years old.

44. The method of any one of the preceding claims, wherein the one or more symptoms or biomarkers that is improved after treatment is bone marrow blasts are substantially absent.

45. The method of any one of the preceding claims, wherein the one or more symptoms or biomarkers that is improved after treatment is bone marrow blasts are less than about 5%.

46. The method of any one of the preceding claims, wherein the one or more symptoms or biomarkers that is improved after treatment is bone marrow blasts and/or peripheral blood blasts are from between 5% to 25%.

47. The method of any one of the preceding claims, wherein the one or more symptoms or biomarkers that is improved after treatment is bone marrow blasts are less than about 10%.

48. The method of any one of the preceding claims, wherein the one or more symptoms or biomarkers after treatment that is improved is bone marrow blasts are less than about 15%.

49. The method of any one of the preceding claims, wherein the one or more symptoms or biomarkers after treatment that is improved is bone marrow blasts are less than about 20%.

50. The method of any one of the preceding claims, wherein the one or more symptoms or biomarkers after treatment that is improved is bone marrow blasts are less than about 25%.

51. The method of any one of the preceding claims, wherein the one or more symptoms or biomarkers that is improved after treatment is peripheral blood blasts are substantially absent.

52. The method of any one of the preceding claims, wherein the one or more symptoms or biomarkers that is improved after treatment is peripheral blood blasts are less than about 5%.

53. The method of any one of the preceding claims, wherein the one or more symptoms or biomarkers that is improved after treatment is peripheral blood blasts are less than about 10%.

54. The method of any one of the preceding claims, wherein the one or more symptoms or biomarkers that is improved after treatment is peripheral blood blasts are less than about 15%.

55. The method of any one of the preceding claims, wherein the one or more symptoms or biomarkers that is improved after treatment is peripheral blood blasts are less than about 20%.

56. The method of any one of the preceding claims, wherein the one or more symptoms or biomarkers that is improved after treatment is peripheral blood blasts are less than about 25%.

57. The method of any one of the preceding claims, wherein the one or more symptoms or biomarkers that is improved after treatment is decrease of pretreatment bone marrow blast percentage by at least 50% based on European Leukemia Net (ELN) 2022 response criteria for AML.

58. The method of any one of the preceding claims, wherein the one or more symptoms or biomarkers that is improved after treatment is the absence of extramedullary disease.

59. The method of any one of the preceding claims, wherein the one or more symptoms or biomarkers that is improved after treatment is absolute neutrophil count (ANC) of 0.5×10⁹/Liters or greater.

60. The method of any one of the preceding claims, wherein the one or more symptoms or biomarkers that is improved after treatment is absolute neutrophil count (ANC) of 1.0×10⁹/Liters or greater.

61. The method of any one of the preceding claims, wherein the one or more symptoms or biomarkers that is improved after treatment is platelet count of 50×10⁹/Liters or greater.

62. The method of any one of the preceding claims, wherein the one or more symptoms or biomarkers that is improved after treatment is platelet count of 100×10⁹/Liters or greater.

63. The method of any one of the preceding claims, wherein after treatment a Measurable Residual Disease (MRD) is less than about 0.1%.

64. The method of claim 63, wherein MRD is measured by flow cytometry of bone marrow cells and/or blood.
